(19) United States
(12) Patent Application Publication
(10) Pub. No.: US 2006/0161135 A1
(43) Pub. Date: Jul. 20, 2006

(54) Ribbed catheter

(76) Inventor: Brian J. VanDerWoude, Portage, MI (US)

Correspondence Address: HOWARD & HOWARD ATTORNEYS, P.C., THE PINEHURST OFFICE CENTER, SUITE #101, 39400 WOODWARD AVENUE, BLOOMFIELD HILLS, MI, 48304-5151, US

(21) Appl. No.: 11/037,687

(22) Filed: Jan. 18, 2005

(51) Int. Cl. A61M 25/00

(52) U.S. Cl. 604/524

(57) ABSTRACT

A catheter for delivering medication from a fluid source to an anatomical site of a patient is provided. The catheter comprises an elongated tube having an outer wall extending between an open proximal end and a closed distal end of the elongated tube and an inner wall coaxially disposed within the outer wall. The inner wall has an inner surface to define a lumen. A plurality of openings are defined in the walls to discharge the fluid from the lumen to the anatomical site. A plurality of ribs having different configurations are disposed on the inner surface to reinforce the catheter to provide kink resistance and to prevent the blockage of fluid flow within the lumen.

### FIELD OF THE INVENTION

The present invention relates generally to a catheter for delivering fluid such as medication to an anatomical site of a patient. More specifically, the present invention relates to the catheter having internal ribs to reinforce the catheter and provide kink resistance to the catheter and to prevent the blockage of fluid flow through the catheter.

### BACKGROUND OF THE INVENTION

A catheter conveys fluid such as medication from a fluid source to an anatomical site of a patient. A typical catheter comprises an elongated tube extending between an open proximal end and a closed distal end. The elongated tube comprises a wall having an inner surface with a lumen bound by the inner surface to convey the fluid from the fluid source. The elongated tube is often defined by two segments, a continuous segment, which is free of any holes or openings, and an infusion segment in which a plurality of openings are defined in the wall. The plurality of openings discharges the fluid from the lumen into the anatomical site. In other words, each of the plurality of openings provides a corridor for the fluid to enter the anatomical site.

When using the catheter, kinking of the elongated tube of the catheter may result in blocking the flow of the fluid through the lumen, much like a garden hose when it becomes kinked. This blockage prevents the fluid from exiting the downstream openings into the anatomical site. Prior art catheters have attempted to overcome the problems associated with kinking by placing a plurality of ribs within the lumen. One such catheter is shown in U.S. Pat. No. 5,593,394 to Kanesaka et al. Here, the catheter includes a wall with an inner surface and a plurality of circumferentially spaced ribs disposed longitudinally along the inner surface. Each of the ribs shares the same, generally rectangular cross-section. Thus, each of the ribs in Kanesaka et al. assumes the same configuration, e.g., size or shape. U.S. Pat. No. 3,720,235 to Schrock shows tubing used in breathing hoses that includes a wall with an inner surface and a plurality of circumferentially spaced ribs disposed longitudinally along the inner surface. In Schrock, each of the ribs shares the same, generally triangular cross-section. Again, as with Kanesaka et al., each of the ribs in Schrock assumes the same configuration, e.g., size or shape. By limiting the ribs to a single configuration in Kanesaka et al. and Schrock, the ability to vary flow characteristics and kink resistance is limited.

### BRIEF SUMMARY OF THE INVENTION AND ADVANTAGES

The present invention provides a catheter for delivering fluid to an anatomical site of a patient. The catheter comprises an elongated tube having proximal and distal ends and at least one opening for delivering the fluid to the anatomical site. The elongated tube has an inner surface defining a lumen for conveying the fluid to the opening. A plurality of ribs are disposed on the inner surface for reinforcing the catheter to provide kink resistance to the catheter and prevent the blockage of fluid flow within the lumen. The plurality of ribs includes at least one rib having a first configuration and at least one rib having a second configuration different than the first configuration.

By utilizing ribs of different configurations, kink resistance can be varied and flow characteristics can be varied during kinked or non-kinked conditions. Likewise, by utilizing ribs of different configurations, adequate kink resistance can be provided, while optimizing flow characteristics between kinked and non-kinked conditions to achieve more uniform fluid delivery.

### BRIEF DESCRIPTION OF THE SEVERAL VIEWS OF THE DRAWINGS

Advantages of the present invention will be readily appreciated as the same becomes better understood by reference to the following detailed description when considered in connection with the accompanying drawings wherein:

FIG. 1 is a perspective view of a catheter of the present invention;

FIG. 2 is a blown-up view of an infusion section of the catheter of FIG. 1;

FIG. 3 is a cross-sectional view of the infusion section of the catheter of FIG. 1 taken along the line 3-3 in FIG. 2 to show an inner wall of the catheter;

FIG. 3A is the cross-sectional view of FIG. 3 illustrating a flow condition of the catheter when kinked;

FIG. 4 is a cross-sectional view of an alternative catheter of the present invention taken along the line 4-4 in FIG. 2;

FIG. 5 is a longitudinal cross-sectional view of the alternative catheter of FIG. 4 taken along the line 5-5 in FIG. 4;

FIG. 6 is a cross-sectional view of a second alternative catheter of the present invention taken along the line 6-6 in FIG. 2;

FIG. 7 is a longitudinal cross-sectional view of the alternative catheter of FIG. 6 taken along the line 7-7 in FIG. 6; and

FIG. 8 is a cross-sectional view of a third altemafive catheter of the present invention taken along the line 8-8 in FIG. 2.

### DETAILED DESCRIPTION OF THE INVENTION

Referring to the Figures, wherein like numerals indicate like or corresponding parts throughout the several views, a catheter 10 for delivering fluid to an anatomical site of a patient is generally shown at 10. It is to be appreciated that the fluid is preferably medication for pain management, but other fluids such as a saline solution, medication for other purposes, and the like could also be used.

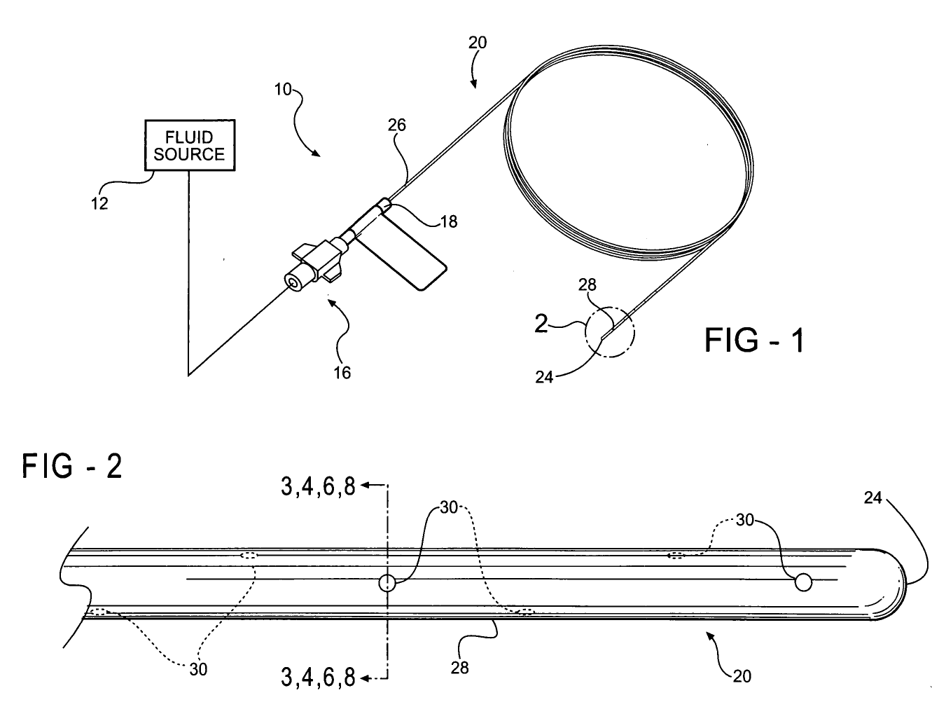

Referring to FIG. 1, the catheter 10 forms part of a fluid delivery system that includes a fluid source generally indicated at 12 to supply the fluid to be delivered to the anatomical site. The fluid source 12 can include any device or manner to store and/or deliver the fluid to the catheter 10. Such devices may include, but are not limited to, pain medication pumps, intravenous bags, tube sets for the same, and the like.

The catheter 10 is in fluid communication with the fluid source 12 and conveys the fluid to the anatomical site. A fluid connection mechanism 16, such as a female luer-lock connector 16, couples the catheter 10 with the fluid source 12. The fluid connection mechanism 16 may also be a threaded fitting, snap fitting, or any type of fitting used by those skilled in the art to couple catheters with fluid sources. It should be appreciated that the catheter 10 may also engage the fluid source 12 without the connector 16.

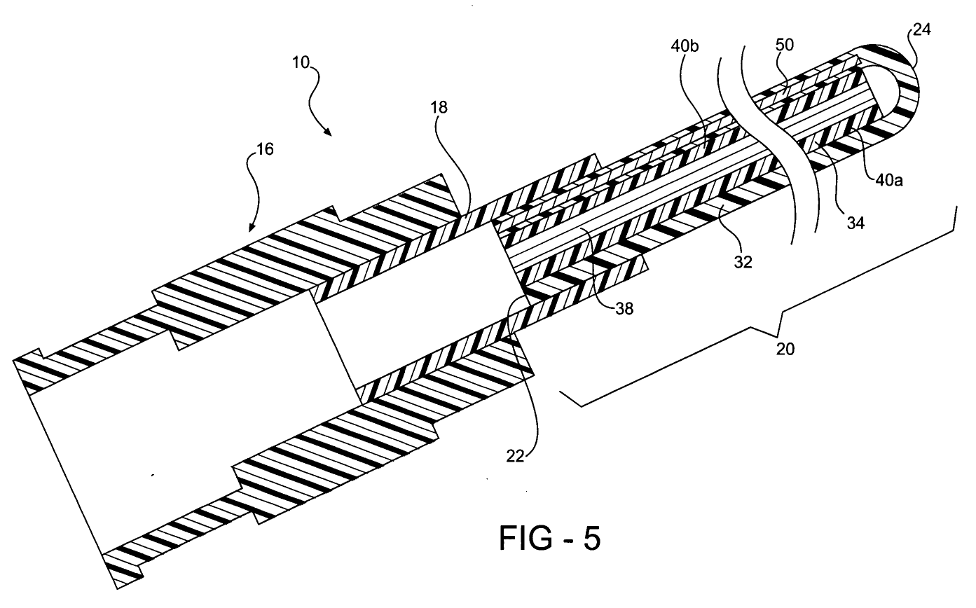

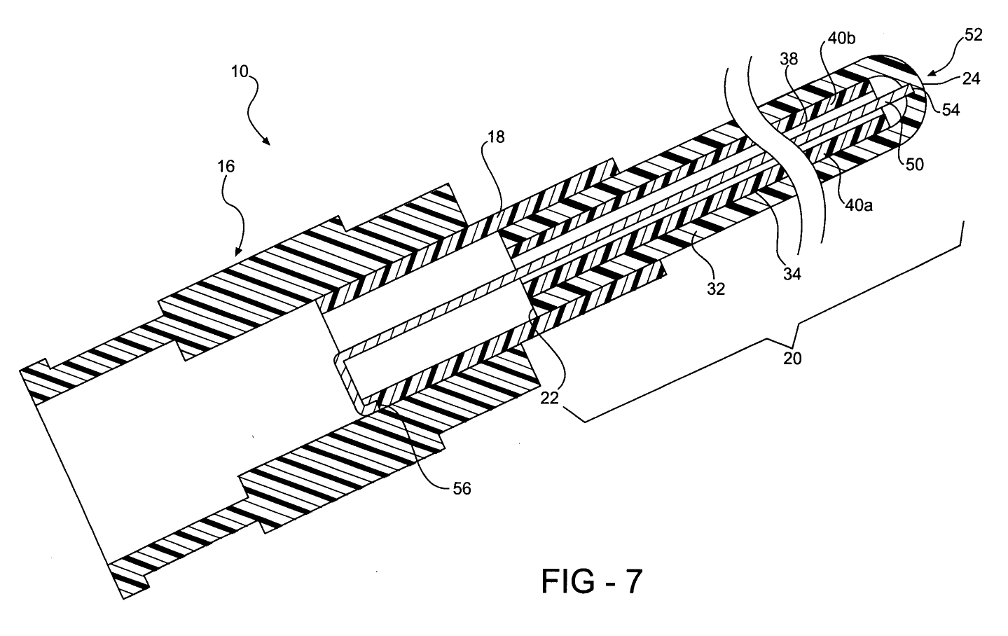

The catheter 10 includes an adapter tube 18 extending from the connector 16 to couple the connector 16 with an elongated tube 20. The adapter tube 18 may be fixed to the connector 16 by any suitable adhesive, weld, and the like. The elongated tube 20 includes an open proximal end 22 partially protruding into the adapter tube 18 (best shown in FIGS. 5 and 7). The proximal end 22 is preferably sealed inside the adapter tube 18 using a suitable adhesive such as a UV curable adhesive, e.g., Dymax® 204 CTH-F. The elongated tube 20 extends from the proximal end 22 inside the adapter tube 18 to a closed distal end 24.

Referring to FIGS. 1 and 2, the elongated tube 20 is shown. The elongated tube 20 may be any length, but in one embodiment is about 36.0 inches in length. The elongated tube 20 includes a continuous segment 26 and an infusion segment 28. These segments merely define boundaries of the elongated tube 20 and do not indicate breaks or connection points in the elongated tube 20. The infusion segment 28 in one embodiment is 5.0 inches in length, but other lengths are possible for various uses of the catheter 10. The continuous segment 26 may include a series of graduated marks to assist a user in determining the depth in which the elongated tube 20 has been inserted into the anatomical site of the patient. The infusion segment 28 includes a plurality of openings 30 that discharge the fluid out from the elongated tube 20 to the anatomical site. The continuous segment 26 is devoid of openings 30, except for the proximal end 22.

Referring to FIG. 2, which illustrates a blown-up view of the infusion segment 28, the openings 30 are preferably axially spaced from adjacent openings 30 along the infusion segment 28 and the openings 30 are also circumferentially spaced about a central axis of the infusion segment 28 by one hundred twenty degrees from adjacent openings 30. Of course, other circumferential spacing of the openings 30 could also be employed. Hidden lines illustrate openings 30 normally not visible in this view. The diameter of each of the plurality of openings 30 preferably ranges from 0.002 to 0.01 inches, more preferably from 0.004 to 0.006 inches. The openings 30 may be micro-drilled or laser-drilled in the elongated tube 20 or formed by other conventional methods.

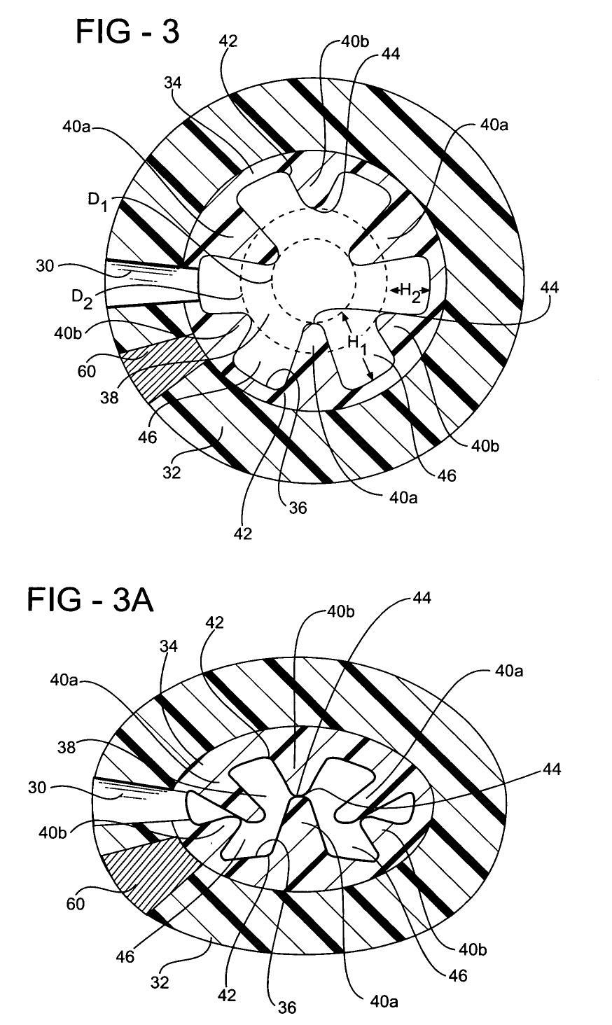

Referring to FIG. 3, the elongated tube 20 may comprise co-extruded outer 32 and inner 34 walls. The inner wall 34 is coaxially disposed within the outer wall 32 and has an inner surface 36 defining a lumen 38 to convey the fluid to the openings 30. The openings 30 are defined through both the outer 32 and inner 34 walls to discharge the fluid from the lumen 38 to the anatomical site.

A plurality of ribs 40 are disposed on the inner surface 36 for reinforcing the catheter 10 to provide kink resistance to the catheter 10 and prevent the blockage of fluid flow within the lumen 38. The plurality of ribs 40 includes a first set 40a of radially and inwardly protruding ribs having a first configuration and a second set 40b of radially and inwardly protruding ribs having a second configuration different than the first configuration. Preferably, the first configuration is further defined as a first peak height H₁ and the second configuration is further defined as a second peak height H₂ that is smaller than the first peak height H₁. In alternative embodiments, the different configurations could be different cross-sectional areas, shapes, sizes, and so on. When the outer 32 and inner 34 walls are kinked, the second set 40b of ribs at least partially supports the first set 40a of ribs, as shown in FIG. 3A, to prevent the blockage of fluid flow through the lumen 38.

Each of the plurality of ribs 40 are disposed longitudinally or lengthwise along the inner surface 36 of the inner wall 34. Preferably, the plurality of ribs 40 are integrally formed with the inner wall 34 to further define the inner surface 36. Each of the plurality of ribs 40 extends between a base 42 and a peak 44 and the peaks 44 of the first set 40a of ribs define a first peak circle D₁ and the peaks 44 of the second set 40b of ribs define a second peak circle D₂ that is greater in diameter than the first peak circle D₁. The plurality of ribs 40 are circumferentially spaced from one another to define channels 46 therebetween. The first set 40a of ribs are alternately arranged with the second set 40b of ribs between the channels 46. Each of the channels 46 is generally U-shaped in cross-section.

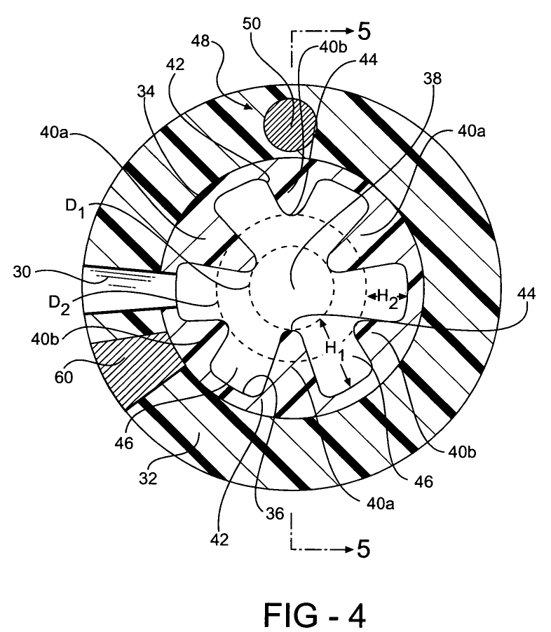

Referring to FIGS. 4 and 5, in one embodiment, a reinforcing member 48 is embedded in the outer wall 32 along a length of the outer wall 32. Preferably, the reinforcing member 48 extends from the proximal end 22 of the elongated tube 20 to just short of the distal end 24, as shown in FIG. 5. It should be understood that the proximal end 22 of the elongated tube 20 and the distal end 24 of the elongated tube 20 also define proximal and distal ends of the outer wall 32. The reinforcing member 48 is further defined as a filament or wire 50. The wire 50 reinforces the outer wall 32 and prevents kinking of the catheter 10 to prevent blockages to fluid flow. The wire 50 may be made from any appropriate sterilizable and biocompatible material, such as 304 stainless steel, titanium, nickel-titanium and plastic filament.

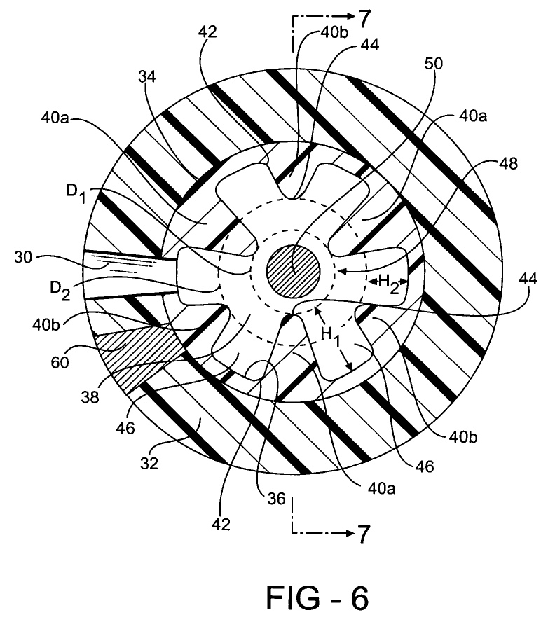

Referring to FIGS. 6 and 7, in another embodiment, the wire 50 is disposed in the lumen 38 between the proximal 22 and distal 24 ends of the elongated tube 20. Here, the elongated tube 20 includes a tip 52 at the distal end 24 and one end of the wire 50 is embedded in the tip 52. More specifically, a distal end 54 of the wire 50 is embedded in the distal end 24 of the elongated tube 20. As will be appreciated by those skilled in the art, the distal end 24 of the elongated tube 20 is formed by radio frequency (RF) tipping techniques and the wire 50 is embedded and fixed in the tip 52 during this process. From the distal end 24, the wire 50 extends proximally and unattached along a length thereof to the connector 16, as shown in FIG. 7. There, a proximal end 56 of the wire 50 is fixed to either the adapter tube 18 or the connector 16. As shown, the proximal end 56 of the wire 50 is embedded in the adapter tube 18. In other embodiments, the proximal end 56 of the wire 50 may be fixed to the adapter tube 18 or the connector 16 with adhesive, or likewise secured between the adapter tube 18 and the connector 16 during assembly.

In each of these embodiments, the outer wall 32 is preferably formed of a first material and the inner wall 34 is formed of a second material different than the first material. In one embodiment, the second material is more rigid than the first material. More specifically, the first material is preferably polyether block amide and the second material is preferably nylon, polyimide, or polyester. Of course, other like materials could be used in alternative embodiments.

Referring back to FIGS. 3, 4, and 6, a radiopaque stripe 60, such as those made of tungsten, may be formed, e.g., extruded, in the outer wall 32 to provide a visual indication as to the location of the catheter 10 within the patient, as is well understood by those skilled in the art. Alternatively, the wire 50 in the previous embodiments may act as the radiopaque stripe provided that the wire 50 is formed of a material of suitable opacity.

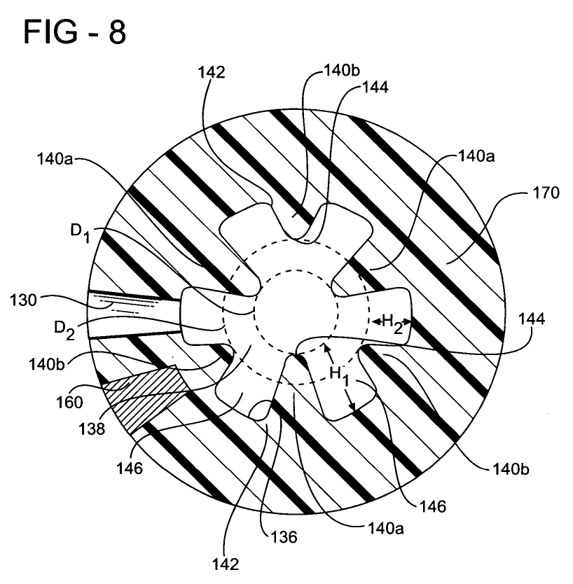

In a further embodiment shown in FIG. 8, in which like numerals are increased by 100, the elongated tube 120 includes a single wall 170 formed of one material, as opposed to having outer and inner walls formed of different materials. Other than this exception, the embodiment of FIG. 8 is identical to the embodiment of FIG. 3. Furthermore, additional embodiments of this single wall version may include the wire 50 of FIGS. 4-5 and 6-7. In other words, additional embodiments are identical to the embodiments of FIGS. 4-5 and 6-7, but with the single wall 170.

Obviously, many modifications and variations of the present invention are possible in light of the above teachings. The invention may be practiced otherwise than as specifically described within the scope of the appended claims.

What is claimed is:

1. A catheter for delivering fluid to an anatomical site of a patient, comprising:
an elongated tube having proximal and distal ends and defining at least one opening for delivering the fluid to the anatomical site;
said elongated tube having an inner surface defining a lumen for conveying the fluid to said at least one opening; and
a plurality of ribs disposed on said inner surface for reinforcing said catheter to provide kink resistance to said catheter and prevent the blockage of fluid flow within said lumen;
wherein said plurality of ribs includes at least one rib having a first configuration and at least one rib having a second configuration different than said first configuration.

2. A catheter as set forth in claim 1 wherein said first configuration is further defined as a first peak height and said second configuration is further defined as a second peak height.

3. A catheter as set forth in claim 2 wherein said plurality of ribs are further defined as a first set of radially and inwardly protruding ribs having said first peak height and a second set of radially and inwardly protruding ribs having said second peak height.

4. A catheter as set forth in claim 3 wherein said second set of ribs at least partially supports said first set of ribs when said elongated tube is kinked to prevent the blockage of fluid flow through said lumen.

5. A catheter as set forth in claim 4 wherein each of said plurality of ribs are disposed lengthwise along said inner surface.

6. A catheter as set forth in claim 5 wherein each of said plurality of ribs extends between a base and a peak and said peaks of said first set of ribs define a first peak circle and said peaks of said second set of ribs define a second peak circle that is greater in diameter than said first peak circle.

7. A catheter as set forth in claim 3 wherein said plurality of ribs are circumferentially spaced from one another to define channels therebetween.

8. A catheter as set forth in claim 7 wherein said first set of ribs are alternately arranged with said second set of ribs between said channels.

9. A catheter as set forth in claim 7 wherein each of said channels is generally U-shaped in cross-section.

10. A catheter as set forth in claim 1 including a reinforcing member embedded in said elongated tube along a length of said elongated tube.

11. A catheter as set forth in claim 10 wherein said reinforcing member is further defined as a wire.

12. A catheter as set forth in claim 1 including a reinforcing member disposed in said lumen along a length of said elongated tube.

13. A catheter as set forth in claim 12 wherein said reinforcing member is further defined as a wire fixed at said proximal end and at said distal end of said elongated tube.

14. A catheter as set forth in claim 13 wherein said elongated tube includes a tip at said distal end and said wire is embedded in said tip.

15. A catheter as set forth in claim 1 wherein said proximal end of said elongated tube is open and said distal end of said elongated tube is closed.

16. A catheter as set forth in claim 1 including a connector coupled to said outer wall at said proximal end for connecting said catheter to a fluid source.

17. A catheter as set forth in claim 1 wherein said at least one opening defined in said elongated tube is further defined as a plurality of openings defined in said elongated tube to discharge the fluid from said lumen and through said elongated tube to the anatomical site.

18. A catheter as set forth in claim 1 including a radiopaque stripe formed in said elongated tube.

19. A catheter for delivering fluid to an anatomical site of a patient, comprising:
an elongated tube having proximal and distal ends and defining at least one opening for delivering the fluid to the anatomical site;
said elongated tube including an outer wall and an inner wall coaxially disposed within said outer wall and having an inner surface defining a lumen for conveying the fluid to said at least one opening; and
a plurality of ribs disposed on said inner surface for reinforcing said catheter to provide kink resistance to said catheter and prevent the blockage of fluid flow within said lumen;
wherein said plurality of ribs includes at least one rib having a first configuration and at least one rib having a second configuration different than said first configuration.

20. A catheter as set forth in claim 19 wherein said first configuration is further defined as a first peak height and said second configuration is further defined as a second peak height.

21. A catheter as set forth in claim 20 wherein said plurality of ribs are further defined as a first set of radially and inwardly protruding ribs having said first peak height and a second set of radially and inwardly protruding ribs having said second peak height.

22. A catheter as set forth in claim 21 wherein said second set of ribs at least partially supports said first set of ribs when said tubes are kinked to prevent the blockage of fluid flow through said lumen.

23. A catheter as set forth in claim 22 wherein each of said plurality of ribs are disposed lengthwise along said inner surface.

24. A catheter as set forth in claim 23 wherein each of said plurality of ribs extends between a base and a peak and said peaks of said first set of ribs define a first peak circle and said peaks of said second set of ribs define a second peak circle that is greater in diameter than said first peak circle.

25. A catheter as set forth in claim 21 wherein said plurality of ribs are circumferentially spaced from one another to define channels therebetween.

26. A catheter as set forth in claim 25 wherein said first set of ribs are alternately arranged with said second set of ribs between said channels.

27. A catheter as set forth in claim 26 wherein each of said channels is generally U-shaped in cross-section.

28. A catheter as set forth in claim 19 including a reinforcing member embedded in said outer wall along a length of said outer wall.

29. A catheter as set forth in claim 28 wherein said reinforcing member is further defined as a wire.

30. A catheter as set forth in claim 19 including a reinforcing member disposed in said lumen between said proximal and said distal ends of said elongated tube.

31. A catheter as set forth in claim 30 wherein said reinforcing member is further defined as a wire fixed at said proximal end and at said distal end.

32. A catheter as set forth in claim 31 wherein said elongated tube includes a tip at said distal end and said wire is embedded in said tip.

33. A catheter as set forth in claim 19 wherein said outer wall is formed of a first material and said inner wall is formed of a second material different than said first material.

34. A catheter as set forth in claim 33 wherein said second material is more rigid than said first material.

35. A catheter as set forth in claim 34 wherein said first material is polyether block amide and said second material is nylon.

36. A catheter as set forth in claim 19 wherein said outer and inner walls are co-extruded.

37. A catheter as set forth in claim 19 wherein said proximal end of said elongated tube is open and said distal end of said elongated tube is closed.

38. A catheter as set forth in claim 19 including a connector coupled to said elongated tube at said proximal end for connecting said catheter to a fluid source.

39. A catheter as set forth in claim 19 wherein said at least one opening defined in said elongated tube is further defined as a plurality of openings defined in both of said walls to discharge the fluid from said lumen and through said walls to the anatomical site.

40. A catheter as set forth in claim 19 including a radiopaque stripe formed in said outer wall.

41. A catheter as set forth in claim 19 wherein said plurality of ribs are integrally formed with said inner wall to further define said inner surface.

42. A catheter, comprising:
an elongated tube having proximal and distal ends and an inner surface defining a plurality of ribs disposed longitudinally therealong for reinforcing said catheter to provide kink resistance to said catheter;
said plurality of ribs including at least one rib having a first configuration and at least one rib having a second configuration different than said first configuration.
